(19) United States
(12) Patent Application Publication
(10) Pub. No.: US 2021/0015964 A1
(43) Pub. Date: Jan. 21, 2021

(54) METHOD OF FORMING AND USING A HEMOSTATIC HYDROCOLLOID

(71) Applicant: United Health Products, Inc., Henderson, NV (US)

(72) Inventors: Sasan Najibi, Encino, CA (US); Douglas Beplate, Bow, WA (US)

(21) Appl. No.: 16/933,746

(22) Filed: Jul. 20, 2020

Related U.S. Application Data
(60) Provisional application No. 62/875,798, filed on Jul. 18, 2019.

(51) Int. Cl. A61L 24/08; A61J 1/20; A61M 39/10; A61M 39/22; A61M 35/00; A61L 24/00

(52) U.S. Cl. CPC A61L 24/08; A61J 1/2089; A61M 39/10; A61B 2017/12004; A61M 35/003; A61L 24/0031; A61M 39/22

(57) ABSTRACT

A method and system for forming a hemostatic hydrocolloid for dispensing into a wound site includes a polymer of oxidized derivatized esterified cellulose in solid form comprising a chain of monomers, wherein, for a first plurality of the monomers in the chain: R is —OCH₂(COO)CH₂CH₃, R1 is —OCH₂(COO)CH₂CH₃, and R2 is —CH₂OCH₂(COO)CH₂CH₃; and wherein, for a second plurality of monomers in the chain: R is —OCH₂(COO)CH₂CH₃, R1 is —OCH₂(COO)CH₂CH₃, and R2 is —(COO)CH₂CH₃ and a liquid mixed with the polymer to form a hemostatic gel for dispensing into a wound site.

### CROSS-REFERENCE TO RELATED APPLICATION

This application claims priority to U.S. Provisional Patent Application No. 62/875,798, filed on Jul. 18, 2019, the entirety of which is incorporated by this reference.

### BACKGROUND OF THE INVENTION

### Field of the Invention

The present invention relates to methods of forming and using a hemostatic material, and more specifically to methods of forming and using a hemostatic hydrocolloid that is formed into a gel or foam used to control bleeding and oozing from a variety of wounds.

### Background and Related Art

Surgical procedures and injuries are often characterized by blood loss. Conventional approaches for dealing with blood loss, such as manual pressure, cauterization, or sutures can be time consuming and are not always effective in controlling bleeding.

A number of topical hemostatic agents have been developed to control bleeding resulting from surgical procedures and injury. Some hemostatic agents, such as collagen-based powders, sponges, and cloths, are of a particulate nature. Such particulate hemostatic agents provide a lattice for natural thrombus formation, but are unable to enhance this process in coagulopathic patients. Pharmacologically-active agents, such as thrombin, can be used in combination with a particulate carrier, for example, as in a gel-foam sponge or powder soaked in thrombin, collagen, and/or calcium. Thrombin has been used to control bleeding on diffusely bleeding tissue surfaces, but the lack of a framework onto which the clot can adhere has limited its use. The autologous and allogenic fibrin glues can cause clot formation, but do not adhere well to wet tissue and have little impact on actively bleeding wounds.

U.S. Pat. No. 8,557,874, the entirety of which is incorporated by reference, discloses hemostatic fabric materials made from chemically treated cellulose, where the hemostatic material is soluble on wound surfaces. In one embodiment, the hemostatic material comprises a polymer of oxidized derivatized esterified cellulose comprising a chain of monomers, each monomer having a structural formula of:

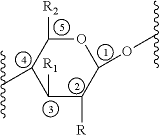

wherein, for a first plurality of the monomers in the chain: R is —OCH₂(COO)CH₂CH₃, R1 is —OCH₂(COO)CH₂CH₃, and R2 is —CH₂OCH₂(COO)CH₂CH₃; and wherein, for a second plurality of monomers in the chain: R is —OCH₂(COO)CH₂CH₃, R1 is —OCH₂(COO)CH₂CH₃, and R2 is —(COO)CH₂CH₃. U.S. Pat. No. 8,557,874 further discloses methods for forming a hemostatic fabric material made from chemically treated plant materials used to enhance the process of coagulation. However, application of such a hemostatic fabric material in certain medical procedures may not be particularly practicable. Accordingly, a method of forming a hemostatic hydrocolloid in a gelatinous or foam-like form and applying the hemostatic hydrocolloid in such a form to control bleeding from a variety of wounds, is desirable.

### SUMMARY OF THE INVENTION

The present invention relates to a method of forming a hemostatic hydrocolloid for use in controlling bleeding from a variety of wounds. In particular, the present invention relates to the formation of a hemostatic hydrocolloid from a solid hemostatic material made from chemically treated plant materials that are soluble in water. The resulting hemostatic hydrocolloid is suitable for controlling active bleeding and oozing.

In some cases, the solid hemostatic material may comprise oxidized derivatized esterified cellulose that is based on a beta-(1-4)-D-glucopyranose polymer of cellulose. From polymers of cellulose, oxidized derivatized esterified cellulose may be created through the oxidation of a hydroxyl group on carbon 6 and/or the derivatization of the hydroxyl group on carbon 2, carbon 3, and/or carbon 6 (if carbon 6 is not oxidized) of monomers within the polymer to form one or more acetic acid esters. In some instances, one or more of the acetic acid esters from carbons 2, 3, and/or 6 of the monomers may then be ethoxylated to form an ethyl ester.

The solid hemostatic material may be in the form of granules, flakes or a powder. The solid hemostatic material is then combined with a liquid, safe for injection into or use on a human body, to form a hydrocolloid in a gelatinous or foam-like form, hereinafter referred to as a hemostatic hydrocolloid. For example, the liquid may comprise sterilized water, saline and/or thrombin or a foaming agent, such as albumin. In addition, the hemostatic hydrocolloid may be combined with other materials and/or substances such antibiotics for additional benefits. The solid hemostatic material may be admixed with the liquid by various methods. For example, the hemostatic hydrocolloid may be formed by connecting two syringes (such as two 10 ML syringes) to a three-way-stopcock between. One syringe contains the solid hemostatic material and the other syringe contains the liquid. The liquid from one syringe is forced through the stopcock or a female-to-female connector into the syringe containing the solid hemostatic material. The liquid and hemostatic material is then passed back and forth from one syringe to the other until a uniform consistency is achieved (e.g., 30 seconds to 2 minutes). The mixing of the solid hemostatic material with the liquid forms a gel that can be injected into a desired position with one of the syringes containing the gel after mixing.

The hemostatic hydrocolloid may be used both outside and inside the body and can be absorbed by the human body. Because of the coagulation properties of the hemostatic hydrocolloid, hemostasis may be fast. Depending upon the nature of the wound and the treatment method employed, the hemostatic hydrocolloid can be fabricated in various forms for controlling the active bleeding from an artery or vein, or for controlling internal bleeding during laparoscopic procedures or surfaces of bone in orthopedic procedures. The hemostatic hydrocolloid can be safely injected into or around tissue to achieve hemostasis. In addition, such a hemostatic hydrocolloid does not present a risk of obstruction or compression of pressure sensitive organs or tissues due to excessive swelling. Such a hemostatic hydrocolloid is specifically applicable in neuro/spine, laparoscopic, orthopedic and cardiovascular surgery.

The solid hemostatic material may be provided in a kit containing two syringes with a first syringe containing the solid hemostatic material in powder or granular form and a second syringe containing the desired liquid. Using a stopcock or a female-to-female connector connected between the two syringes, the solid hemostatic material can be mixed with the liquid to form a gel at the time when the hemostatic hydrocolloid is needed. Alternatively, the hemostatic hydrocolloid is provided in a pre-made gelatinous form in a single syringe. In yet another embodiment, the hemostatic hydrocolloid is provided within a pressurized container containing a liquid foaming agent so that a foamed hemostatic material can be dispensed as needed. Thus, regardless of the form in which the hemostatic material is provided, it is easy to carry and store, is stable, has a relatively long shelf life, meets the requirements of surgery and daily use, can be applied for emergency hemostasis in the battle ground, causes no pain, conforms to wounds accurately, adheres well to wound sites, even when wet, has no known side effects, and exhibits high hemostasis efficacy-even in patients with a blood-coagulation defect or patients on blood-thinning drugs. The hemostatic hydrocolloid of the invention is simple to form, safe and easy to use, economical, can be utilized under any circumstances where hemostasis is needed and can be made economically in the industry.

In one embodiment, a method of forming a hemostatic hydrocolloid for dispensing into a wound site comprises providing a polymer of oxidized derivatized esterified cellulose in solid form in a first mixing and dispensing device, the polymer of oxidized derivatized esterified cellulose comprising a chain of monomers, wherein, for a first plurality of the monomers in the chain: R is —OCH₂(COO)CH₂CH₃, R1 is —OCH₂(COO)CH₂CH₃, and R2 is —CH₂OCH₂(COO)CH₂CH₃; and wherein, for a second plurality of monomers in the chain: R is —OCH₂(COO)CH₂CH₃, R1 is —OCH₂(COO)CH₂CH₃, and R2 is —(COO)CH₂CH₃. A liquid is provided in a second mixing and dispending device. The first mixing and dispending device is connected to the second mixing and dispensing device to allow flow of the liquid between the first and second dispensing and mixing devices. The liquid is dispensed into the polymer of oxidized derivatized esterified cellulose in solid form. The liquid and polymer of oxidized derivatized esterified cellulose are repeatedly mixed by passing them between the first and second mixing and dispensing devices until a hemostatic gel is formed.

In another embodiment, an adapter is connected between the first and second mixing and dispensing devices, the adapter is configured to allow flow of the liquid between the first and second mixing and dispensing devices.

In one embodiment, the adapter comprises a three-way stopcock. In another embodiment, the adapter comprises a female-to-female luer lock adapter.

In another embodiment, the stopcock includes a valve that is positioned so that flow can pass only between the first and second mixing and dispensing devices.

The liquid may comprise sterile water or saline.

The polymer of oxidized derivatized esterified cellulose may be in the form of granules, particles, pieces or in granulated or powdered form.

In one embodiment, the first dispensing and mixing device comprises a first syringe and the second dispensing and mixing device comprises a second syringe.

In another embodiment, pressing a second plunger of the second syringe forces the liquid from the second syringe into the first syringe, thereby causing a first syringe plunger of the first syringe to extend and wetting the polymer of oxidized derivatized esterified cellulose to form a hemostatic material/liquid mixture.

In yet another embodiment, pressing the second syringe plunger forces the hemostatic material/liquid mixture from the second syringe into the first syringe and causing the first syringe plunger of the first syringe to extend.

In still another embodiment, pressing the first syringe plunger forces the hemostatic material/liquid mixture from the first syringe into the second syringe and causing the second syringe plunger of the second syringe to extend and repeatedly alternating pressing the second syringe plunger and first syringe plunger until a visibly homogenous mixture of the hemostatic material/liquid is formed, resulting in the formation of a hemostatic gel.

In another embodiment, the first or second syringe that contains the hemostatic gel is removed from the adapter and the selected syringe is used to inject the hemostatic gel into a wound site to inhibit bleeding.

In one embodiment, a system for forming a hemostatic hydrocolloid, comprises a polymer of oxidized derivatized esterified cellulose comprising a chain of monomers, wherein, for a first plurality of the monomers in the chain: R is —OCH₂(COO)CH₂CH₃, R1 is —OCH₂(COO)CH₂CH₃, and R₂ is —CH₂OCH₂(COO)CH₂CH₃; and wherein, for a second plurality of monomers in the chain: R is —OCH₂(COO)CH₂CH₃, R1 is —OCH₂(COO)CH₂CH₃, and R2 is —(COO)CH₂CH₃; and a water-based liquid mixed with the polymer of oxidized derivatized esterified cellulose forming a hemostatic gel.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates some embodiments of the chemical formula of a cellulose polymer;

FIG. 2 illustrates some embodiments of the chemical formula of a monomer that may be used to form polymers of the hemostatic material of the invention;

FIG. 3 illustrates some embodiments of the chemical formula of a portion of a polymer that may be used in the hemostatic material of the invention;

FIG. 4 illustrates some embodiments of the chemical formula of a portion of a polymer that may be used in the hemostatic material of the invention;

FIG. 5 illustrates some embodiments of the chemical formula of a monomer that may be used to form polymers of the hemostatic material of the invention;

FIG. 6 illustrates a first embodiment of a system for mixing a hemostatic material with a liquid in accordance with the principles of the present invention;

FIG. 7A illustrates a second embodiment of a system for mixing a hemostatic material with a liquid in accordance with the principles of the present invention;

FIG. 7B illustrates an adapter of the system for mixing a hemostatic material with a liquid shown in FIG. 7A.

FIG. 8 illustrates a system for dispensing a liquid hemostatic material in accordance with the principles of the present invention;

FIG. 9 illustrates a container for dispensing a hemostatic material in the form of a foam in accordance with the principles of the present invention.

### DETAILED DESCRIPTION OF THE INVENTION

The present invention relates to a method of forming a hemostatic hydrocolloid in a gelatinous or foamed form that is bioabsorbable and can be formed on site for use in controlling bleeding from a variety of wounds. In particular, the present invention relates to hemostatic hydrocolloids made from chemically treated plant materials, such as cellulose, that are made to be water soluble. The hemostatic hydrocolloids are suitable for controlling active bleeding and oozing from tissues.

1. Hemostasis

To better explain the hemostatic hydrocolloid of the invention, a non-binding description of hemostasis is provided herein. The term hemostasis may be used to refer to the mechanism (e.g., normal vasoconstriction, abnormal obstruction, coagulation, or surgical means) that stems bleeding after injury to the vasculature. Biological hemostasis depends on both cellular components and soluble plasma proteins. In particular, hemostasis by coagulation may be dependent upon a complex interaction of plasma coagulation and fibrinolytic proteins, platelets, and the blood vasculature. The hemostatic process may be conceptually separated into three stages: primary hemostasis, secondary hemostasis, and tertiary hemostasis.

Primary hemostasis may principally be characterized by the formation of a primary platelet plug. The plug may be formed as circulating platelets adhere and aggregate at sites of blood vessel injury. In areas of high shear rate (e.g., microvasculature) aggregation may be mediated by von Willebrand factor (vWf), which may bind to glycoprotein Ib-IX in the platelet membrane. In areas of low shear rate (e.g., arteries) fibrinogen mediates the binding of platelets to the subendothelium by attaching to a platelet receptor. Aggregation begins with platelets adhering to exposed subendothelium. When platelets adhere to the vessel wall, they change shape and activate the collagen receptor on their surface to release alpha and dense granule constituents. Injury to the blood vessel wall is additionally followed by vasoconstriction. Vasoconstriction not only retards extravascular blood loss, but also slows local blood flow, enhancing the adherence of platelets to exposed subendothelial surfaces and the activation of the coagulation process.

Formation of the plug may be followed by an aggregation response. Activation of platelets results in exposure of anionic phospholipids that serve as platforms for the assembly of blood coagulation enzyme complexes. Platelet aggregation involves the activation, recruitment, and binding of additional platelets to the adhered platelets. Aggregation is promoted by platelet agonists, such as thromboxane 2, PAF, ADP, and serotonin. Activated platelets synthesize and release thromboxane and platelet activating factor, which are potent platelet aggregating agonists and vasoconstrictors. Activation is enhanced by the generation of another platelet agonist, thrombin, through the coagulation cascade. Platelet aggregation is mediated primarily by fibrinogen, which binds to glycoprotein IIb/IIIa on adjacent platelets. This aggregation leads to the formation of the primary platelet plug, and is stabilized by the formation of fibrin.

Secondary hemostasis may be characterized by fibrin formation through the coagulation cascade, which involves circulating coagulation factors, calcium, and platelets. The coagulation cascade involves three pathways: intrinsic; extrinsic; and common. The main pathway for initiation of coagulation is the extrinsic pathway, while the intrinsic pathway acts to amplify the coagulation cascade.

The extrinsic pathway may involve the tissue factor and factor VII complex, which activates factor X. The extrinsic pathway of blood coagulation is initiated when blood is exposed to tissue factor. Tissue factor, a transmembrane protein, is expressed by endothelial cells, subendothelial tissue and monocytes, with expression being upregulated by cytokines. Tissue factor binds activated factor VII (factor Vila) and the resulting complex activates factors X and IX. Factor X, in the presence of factor V, calcium, and platelet phospholipid, then activates prothrombin to thrombin. This pathway is rapidly inhibited by a lipoprotein-associated molecule referred to as tissue factor pathway inhibitor. However, the small amount of thrombin generated by this pathway activates factor XI of the intrinsic pathway, which amplifies the coagulation cascade.

Thrombin activates the intrinsic pathway by activation of factors XI and VIII. In the intrinsic pathway activated factor IX (factor IXa) combines with factor Villa to provide a second means to activate factor X. The intrinsic pathway involves high-molecular weight kininogen, prekallikrein, and factors XII, XI, IX and VIII. Factor VIII acts as a cofactor (with calcium and platelet phospholipid) for the factor IX-mediated activation of factor X. Activated factor IX, together with activated factor VIII, calcium, and phospholipid, referred to as tenase complex, amplify the activation of factor X, generating large amounts of thrombin.

The extrinsic and intrinsic pathways converge at the activation of factor X. The common pathway involves the factor X-mediated generation of thrombin from prothrombin (facilitated by factor V, calcium and platelet phospholipid), with the production of fibrin from fibrinogen. Factor Xa complexes with factor Va and prothrombin to form prothrombinase, which cleaves prothrombin to generate thrombin, the key enzyme in hemostasis. In the final step of the coagulation cascade, thrombin cleaves fibrinogen to generate fibrin monomers, which then polymerize. This polymer is covalently cross-linked by factor XIIIa (itself generated from factor XIII by thrombin) to form a chemically stable clot. Thrombin also feeds back to activate cofactors V and VIII, thereby further amplifying the coagulation system.

Tertiary hemostasis is characterized by the formation of plasmin, which is the main enzyme responsible for fibrinolysis. At the same time as the coagulation cascade is activated, tissue plasminogen activator is released from endothelial cells. Tissue plasminogen activator binds to plasminogen within the clot, converting it into plasmin. Plasmin lyses both fibrinogen and fibrin in the clot, releasing fibrin and fibrinogen degradation products.

Finally, fibrin is digested by the fibrinolytic system, the major components of which are plasminogen and tissue-type plasminogen activator (tPA). Both of these proteins are incorporated into polymerizing fibrin, where they interact to generate plasmin, which, in turn, acts on fibrin to dissolve the preformed clot.

The fibrinolytic system is, in turn, regulated by three serine proteinase inhibitors, namely, antiplasmin, plasminogen activator inhibitor-1 (PAI-1), and plasminogen activator inhibitor-2 (PAI-2). Plasma D-dimers are generated when the endogenous fibrinolytic system degrades fibrin. They consist of two identical subunits derived from two fibrin molecules. Unlike fibrinogen degradation products, which are derived from fibrinogen and fibrin, D-dimers are a specific cross-linked fibrin derivative

The process of fibrin deposition is limited by mechanisms of the natural anticoagulant system. The maintenance of adequate blood flow and the regulation of cell surface activity limit the local accumulation of activated blood coagulation enzymes and complexes. Antithrom bin (AT) is a plasma protein member of the serpin (serine protease inhibitor) family that inhibits the activities of all of the activated coagulation enzymes. The inhibitory effect of AT is increased several thousand-fold by binding to heparin. Protein C is a vitamin K-dependent protein that proteolyses factor Va and factor Villa to inactive fragments. Protein C binds to an endothelial cell protein C receptor (EPCR) and is activated by thrombin bound to thrombomodulin, another endothelial cell membrane-based protein, in a reaction that is modulated by a cofactor, protein S. Tissue factor pathway inhibitor is a lipoprotein-associated plasma protein that forms a quaternary complex with tissue factor, factor Vila, and factor X, thereby inhibiting the extrinsic coagulation pathway.

2. Hemostatic Mechanism

The following is a description of the ways in which the hemostatic hydrocolloid of the invention may contribute to achieving hemostasis:

a) Hemostasis Through Physical Path

When the hemostatic hydrocolloid contacts blood, the hemostatic material may stimulate a blood clotting cascade. For example, the hemostatic material, which has absorbed a (e.g., water, saline or thrombin) and thereby changed from a solid to a gelatinous substance, can slow the flow of the blood. The soluble hemostatic gel may cover the wound surfaces and further expand after as it absorbs fluid. As the hemostatic gel contacts fluid in the blood, some part of the hemostatic material may form a viscous body and clog the end of capillary blood vessels.

b) Hemostasis Through Chemical Path

The term “Hemostasis through chemical path” means that when the hemostatic hydrocolloid of the invention contacts platelets, absorption and coagulation may occur at an increased rate.

c) Hemostasis Through Physiology Path

The term “Hemostasis through physiology path” means that the hemostatic hydrocolloid of the invention can activate the coagulation factors in the human body and boost the formation of thrombin so as to generate hemostasis efficacy. The coagulation factor may be the key factor to activate the endogenous coagulation system as well as the external coagulation system. It is already known that some coagulation factors may bring positive electricity; therefore, they could be generally activated by a substance with negative electricity. Because the hemostatic hydrocolloid may be water-soluble, it can generate large quantities of negative electricity to activate the coagulation factors.

3. Hemostatic Hydrocolloid

The various embodiments provide compositions and materials that react with the hemostatic system to treat or prevent bleeding. The compositions and materials of the embodiments may result in coagulation of blood. In particular, the compositions and materials are combined to form a hemostatic hydrocolloid. A hydrocolloid is a substance that forms a gel in the presence of water. In the present invention, the hemostatic hydrocolloid is formed by combining a solid hemostatic, such as a chemically treated, cellulose based material with a liquid, such as water, saline or thrombin.

Effective delivery of hemostatic agents to wounds is desirable in the treatment of injuries characterized by bleeding, as well as in surgical procedures where the control of bleeding can become problematic (e.g., surgical procedures involving large surface areas, heavy arterial or venous bleeding, oozing wounds, organ laceration/resectioning, etc.). The compositions and materials of invention can possess a number of advantages in delivery of hemostatic agents to wounds, including, but not limited to, ease of application and removal, bioadsorption potential, antigenicity, and tissue reactivity.

Depending upon the nature of the wound and the treatment method employed, the embodiments of the hemostatic hydrocolloid can be fabricated in various forms. For example, a gel or a foam when applied to a wound can control active bleeding from an artery or vein, or control internal bleeding during laparoscopic procedures. In neurosurgery, where oozing brain wounds are commonly encountered, the gel or foam can be applied where other solid forms of hemostatic material may be difficult to apply. Despite differences in delivery and handling characteristics of the various forms, the hemostatic hydrocolloid may be effective in deploying hemostatic agents to an affected site and rapidly initiating hemostatic plug formation through platelet adhesion, platelet activation, and/or blood coagulation.

The hemostatic hydrocolloid of the invention may be formed from any appropriate material. For instance, some non-limiting examples of solid materials that may be used to produce the hemostatic hydrocolloid may comprise cellulose, cellulose derivatives (e.g. alkyl cellulose (e.g., methyl cellulose), hydroxyalkyl cellulose, alkylhydroxyalkyl cellulose, cellulose sulfate, salts of carboxymethyl cellulose, carboxymethyl cellulose, and carboxyethyl cellulose), chitin, carboxymethyl chitin, hyaluronic acid, salts of hyaluronic acid, alginate, alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, heparin, heparin sulfate, heparan, heparan sulfate, dermatan sulfate, keratin sulfate, carrageenans, chitosan, starch, amylose, amylopectin, poly-N-glucosamine, polymannuronic acid, polyglucuronic acid, polyguluronic acid and derivatives of the above. The solid materials are combined with a liquid that is safe for human use, such as sterile water, saline and/or thrombin, to form a gelatinous hemostatic material.

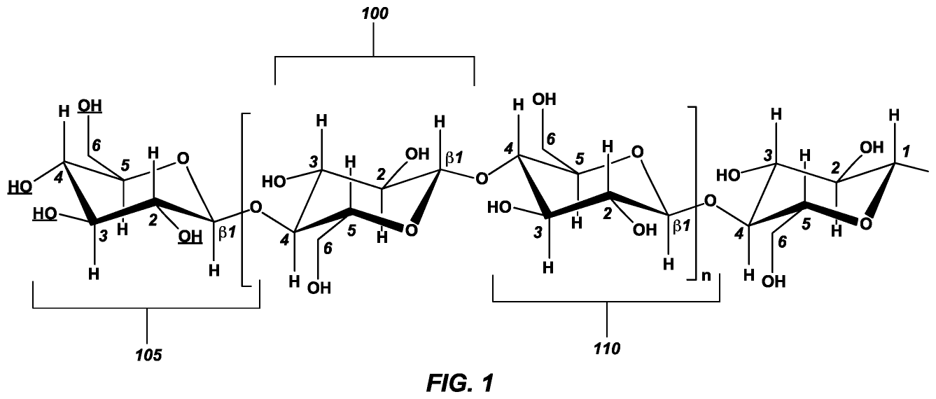

According to some embodiments, however, the described hemostatic hydrocolloid may be based on a beta-(1-4)-D-glucopyranose polymer of cellulose, as is illustrated in FIG. 1. In particular, FIG. 1 illustrates that a beta-linked glucopyranose residue 100, which is connected to two other glucopyranose residues (e.g., residues 105 and 110) in a ⁴C₁ chair configuration, may comprise a hydroxyl group that is bound to carbon 2, carbon 3, and carbon 6.

In some embodiments, the hemostatic hydrocolloid may comprise oxidized derivatized esterified cellulose. According to some embodiments, the hemostatic hydrocolloid comprising oxidized derivatized esterified cellulose may be created from beta-(1-4)-D-glucopyranose polymers through the oxidation of the hydroxyl group on carbon 6 and/or the derivatization of the hydroxyl group on carbon 2, carbon 3, and/or carbon 6 (if carbon 6 is not oxidized) to form one or more acetic acid esters. According to some embodiments, one or more of the acetic acid esters from carbons 2, 3, and/or 6 may then be ethoxylated to form an ethyl ester.

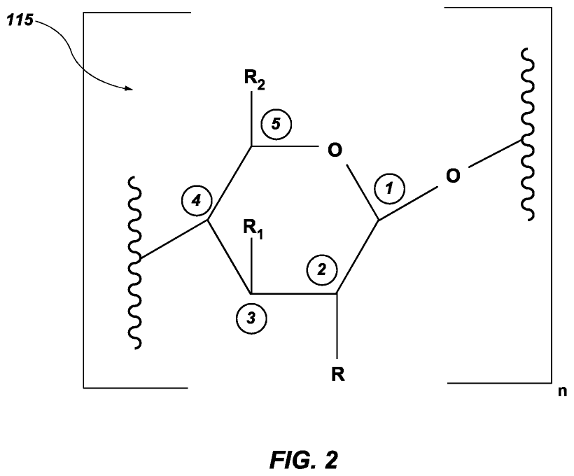

FIG. 2 illustrates some embodiments of the structural formula of a basic unit, or a monomer, of oxidized derivatized esterified cellulose that may be used to create polymers of the hemostatic hydrocolloid. Specifically, FIG. 2 shows that a six-membered glucopyranosyl ring 115 that serves as a monomer of the hemostatic hydrocolloid may comprise a variety of functional groups. For example, FIG. 2 illustrates that a first functional group R may be bound to the ring 115 through carbon 2, a second functional group R₁ may be bound to the ring 115 through carbon 3, and/or a third functional group R₂ may be bound to the ring 115 through carbon 5.

The various functional groups, including R, R₁, and R₂, may comprise any functional group that allows the hemostatic material to be bioabsorbable and to reduce bleeding in a wound. For instance, R, R₁, and/or R₂ may each individually comprise —CH₂OCH₂(COO)CHₓCHₓ, —(COO)CHₓCHₓ, —OCH₂(COO)CHₓCHₓ, —OH, —CH₂OH, or COOH. However, according to some preferred embodiments, R may comprise —OH or an ethyl carboxymethyl group, such as —OCH₂(COO)CH₂CH₃. In some preferred embodiments, R₁ may comprise —OH or an ethyl carboxymethyl group, such as —OCH₂(COO)CH₂CH₃. Moreover, in some embodiments, R₂ may comprise an ethyl carboxymethyl group, such as —CH₂OCH₂(COO)CH₂CH₃, or a carboxyethyl group, such as —(COO)CH₂CH₃.

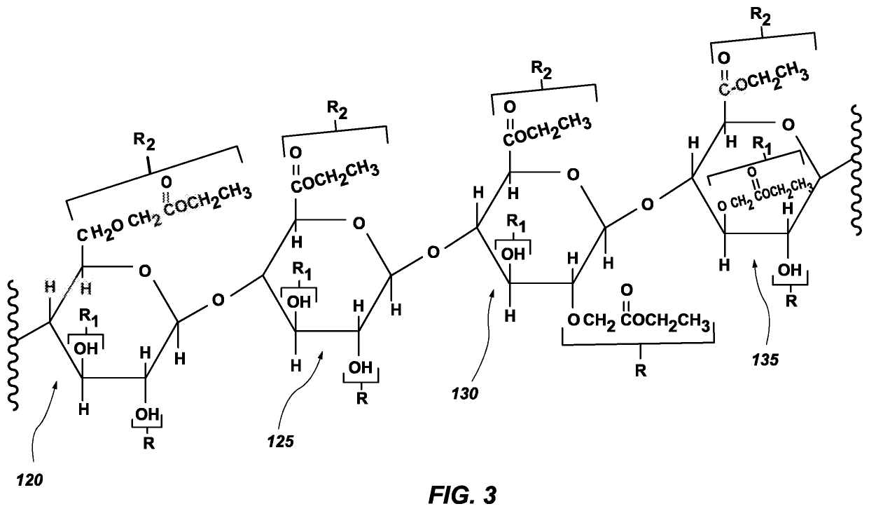

A monomer used to form polymers of the hemostatic hydrocolloid may have any suitable combination of functional groups that allows the hemostatic material to comprise polymers of oxidized derivatized esterified cellulose, and not solely unreacted cellulose. For instance, FIG. 3 illustrates some embodiments of a portion of a polymer of oxidized derivatized esterified cellulose. Namely, FIG. 3 illustrates several non-limiting examples of monomers connected together, wherein each monomer comprises a different combination of functional groups. For example, FIG. 3 illustrates that in a first monomer 120, R may comprise —OH, R₁ may comprise —OH, and R₂ may comprise —CH₂OCH₂(COO)CH₂CH₃. FIG. 3 also shows that in a second monomer 125, R may comprise —OH, R₁ may comprise —OH, and R₂ may comprise —(COO)CH₂CH₃. Further, FIG. 3 depicts that in a third monomer 130, R may comprise —OCH₂(COO)CH₂CH₃, R₁ may comprise —OH, and R₂ may comprise —(COO)CH₂CH₃. Finally, FIG. 3 shows that in a fourth monomer 135, R may comprise —OH, R₁ may comprise —OCH₂(COO)CH₂CH₃, and R₂ may comprise —(COO)CH₂CH₃.

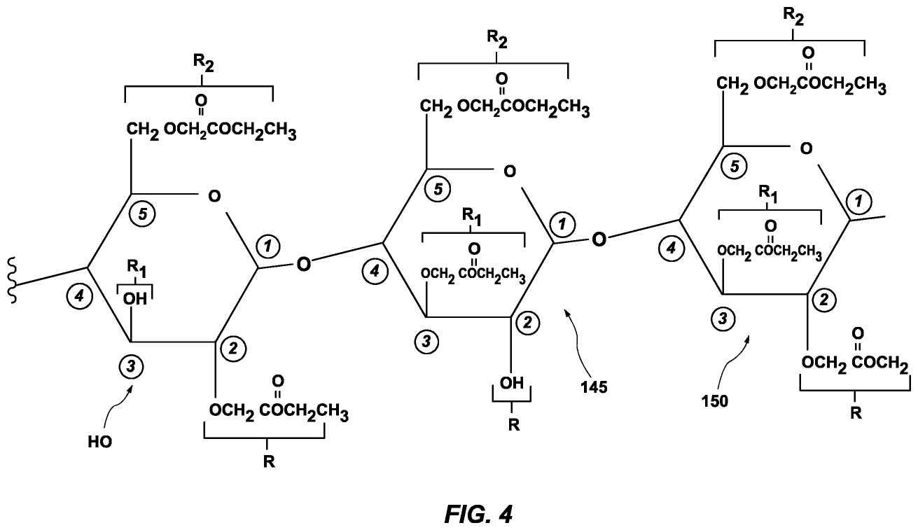

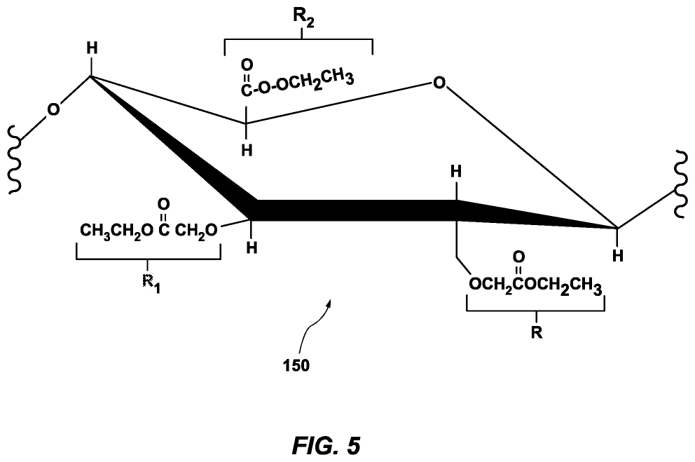

According to some embodiments, FIGS. 4 and 5 illustrate several additional monomers that show other non-limiting examples of functional group combinations that may be possible in a polymer of oxidized derivatized esterified cellulose that is used to form the hemostatic hydrocolloid. Specifically, FIG. 4 shows that in a fifth example of a monomer 140, R may comprise —OCH₂(COO)CH₂CH₃, R₁ may comprise —OH, and R₂ may comprise —CH₂OCH₂(COO)CH₂CH₃. FIG. 4 illustrates that in a sixth example of a monomer 145, R may comprise —OH, R₁ may comprise —OCH₂(COO)CH₂CH₃, and R₂ may comprise —CH₂OCH₂(COO)CH₂CH₃. Also, FIG. 4 depicts a seventh example of a monomer 150 that shows that R may comprise —OCH₂(COO)CH₂CH₃, R₁ may comprise —OCH₂(COO)CH₂CH₃, and R₂ may comprise —CH₂OCH₂(COO)CH₂CH₃. Moreover, FIG. 5 illustrates an eighth non-limiting example of a monomer 150, where the monomer 150 is depicted in a chair configuration. FIG. 5 shows that, in some embodiments, R may comprise —OCH₂(COO)CH₂CH₃, R₁ may comprise —OCH₂(COO)CH₂CH₃, and R₂ may comprise —(CLL)CH₂CH₃.

A polymer of oxidized derivatized esterified cellulose may comprise any combination of monomers that comprise any suitable combination of the aforementioned functional groups. Thus, a monomer with any combination of functional groups may be connected to one or two other monomers with the same or different functional groups located on the same and/or different carbons. For example, FIG. 3 depicts one possible combination of monomers (i.e., monomers 120, 125, 130, and 135) in a portion of a polymer of the hemostatic hydrocolloid. Nevertheless, in other embodiments, monomers with different or similar combinations of functional groups may be connected throughout a polymer of the hemostatic hydrocolloid in any other order that is chemically feasible.

Polymers of the oxidized derivatized esterified cellulose may be any suitable length that allows the polymers to be used to control bleeding. For example, FIG. 2 illustrates that a polymer of oxidized derivatized esterified cellulose may comprise any suitable number of monomers, where the number of monomers is referred to as n. Indeed, in some embodiments, a polymer of oxidized derivatized esterified cellulose may comprise between about 2 and about 150,000 monomers. In other embodiments, however, a polymer may comprise between about 2 and about 20,000 monomers. In still other embodiments, a polymer may comprise between about 500 and about 2,000 monomers. Indeed, in another embodiment, a polymer may comprise about 1000 monomers.

According to some embodiments, the monomers and other compounds may have asymmetric centers. Unless otherwise indicated, all chiral, diastereomeric, and racemic forms of the described monomers and all geometric isomeric forms of the described monomers may be included in the present invention. It will also be appreciated that compounds of the present invention that contain asymmetrically substituted carbon atoms may be isolated in optically active or racemic forms. Furthermore, this invention may encompass any or all intermediate products and byproducts that may be present in the formation of the described oxidized derivatized esterified cellulose. For instance, while in some embodiments, a polymer may be about 99.99% ethoxylated, this invention may comprise polymers that are ethoxylated to higher or lower degrees. Additionally, this invention may also encompass monomers that are connected to other monomers through various forms of ether bonds, which may include beta and/or alpha bonds.

As mentioned, the hemostatic hydrocolloid is water soluble and may be based on a chemically treated plant fiber, such as cellulose. The untreated plant fiber can absorb water, but may be insoluble. After being treated by the process of the invention, its physical and chemical properties are changed significantly so that the resulting hemostatic hydrocolloid is soluble in water and body fluids. As mentioned, the hemostatic hydrocolloid of the invention can be used both inside and outside the body to stop bleeding. When utilized in biological systems the soluble hemostatic material of this invention may continue to absorb water and further expand.

The hemostatic hydrocolloid is provided in the form of a gel or foam, which has been pre-wetted with a liquid, such as sterile water, saline or thrombin. The hemostatic hydrocolloid is formed from a solid hemostatic material that is combined with the liquid and mixed for a period of time until the solid hemostatic material absorbs the liquid and forms a gel. The solid hemostatic material can be provided in the form of, particles, pieces, granules of a pre-selected size or a powder. The resulting hemostatic gel, while being provided with sufficient liquid to be flowable, as through a syringe, has the ability to further absorb other liquid, such as blood when applied to a wound to activate blood-coagulation. The hemostatic hydrocolloid of the invention may increase hemostatic efficacy by at least three mechanisms: physical, chemical, and physiological; each of which are discussed below at greater length. In particular, the hemostatic hydrocolloid may activate the blood-coagulation factors to boost the formation of thrombin, and the material may absorb fluid from the blood and further expand. Application of the hemostatic hydrocolloid may increase the viscosity of blood, blood flow speed may be reduced, and the colloid may clog the opening of the blood vessel through which bleeding is taking place. Because the soluble hemostatic material may activate the blood-coagulation factors and boost the formation of thrombin, it may notably be effective for patients with blood-coagulation obstructions or defects.

The hemostatic hydrocolloid can be used both for a broad range of uses, including clinical and for first aid. It can advantageously and easily be use in hostile environments where a simple and effective means for stopping the flow of blood or body fluids is desired (e.g., battleground situations). The hemostatic hydrocolloid may be soluble and may be used on wound surfaces under pressure. The material can be provided free of any medications, if desired, or may contain desired medications for particular purposes.

The hemostatic hydrocolloid may suitable for use in both surgical applications as well as for use in field treatment of traumatic injuries. For example, the material may be suitable for use in vascular surgery, where bleeding can be particularly problematic. The hemostatic material may be suitable for use in cardiac surgery, where multiple vascular anastomoses and cannulation sites, complicated by coagulopathy induced by extracorporeal bypass, can result in bleeding that can only be controlled by topical hemostats. The hemostatic material may be suitable to produce rapid and effective hemostasis during spinal surgery, where control of osseous, epidural, and/or subdural bleeding or bleeding from the spinal cord is not amenable to sutures or cautery. In such instances, the hemostatic material can minimize the potential for injury to nerve roots and reduce the procedure time. In another example, the hemostatic material may also be suitable for use in liver surgery, in live donor liver transplant procedures, or in the removal of cancerous tumors; where there is a substantial risk of massive bleeding. The material may be suitable for use as an effective hemostatic material, which can significantly enhance patient outcome in such procedures. Even in situations where massive bleeding is not a problem, the hemostatic hydrocolloid may be suitable for use to achieve hemostasis. For example, the material may be used in dental procedures, such as tooth extractions; for abrasions; burns; sports related injuries, and the like. The material may also be suitable for use in neurosurgery, where oozing wounds are common and are difficult to treat.

The nature of the hemostatic material of this invention may include any combination of the following attributes:

a) Water-Solubility

The known prior art cellulose fiber materials may contain hydrophilic hydroxyamino-. However, in such cellulose fiber materials large quantities of hydrogen bonds may exist among the molecules and the degree of crystallinity may be high. Thus, the known prior art cellulose fiber material may not be dissolved in water. Nevertheless, during the processing according to the invention, the cellulose may be chemically changed so that:

i) The degree of polymerization may be decreased, as well as the dispersion force and inductive capacity.

ii) Hydrophilic radical groups may be induced to widen the space between the molecules and destroy the hydrogen bonds inside and/between some of the molecules.

iii) The degree of crystallinity may be decreased, the amorphism zone may be enlarged, orientation forces between molecules may be decreased, and it is possible that water molecules may form molecular compounds in tiny packs.

From the point of view of thermodynamics, the free energy of mixing between the molecules of the hemostatic material and water molecules may be below zero. Furthermore, because the solubility difference may also be less than about 1.7 to about 2.0, dissolution may occur. As mentioned, the dissolution process of the described hemostatic material by water may include: the hemostatic material absorbing fluid and expanding, the unbinding of the structure so the material may be transformed to a gel, and dissolving the material completely.

b) Absorbability to Water and Polarizable Medium

If the speed of absorption of the hemostatic material to water and polarizable medium is high, the amount of absorption may be large. This can be helpful for hemostasis.

4. Method for Making Soluble Hemostatic Material for use as a Hemostatic Gel

The formation of the soluble hemostatic material comprised of oxidized derivatized esterified cellulose may be accomplished in any suitable manner. Nevertheless, in order to provide a better understanding of the soluble hemostatic material and its methods of production, a non-limiting example of a typical method of making the hemostatic material is provided below, wherein the method may comprise:

a) Placing sodium hydroxide, sodium carbonate, sodium hypochlorite in to the internal bladder of a reaction vessel, then adding in an appropriate amount of pure water and stirring until the ingredients are dissolved. Pouring ethyl alcohol (preferably about 95% ethyl alcohol) in to solution in the internal bladder and mixing. Turning on a heater and keeping the temperature of the internal bladder above 20° C. (preferably between about 25° C. and about 28° C.) and holding at the desired temperature for a period of time, preferably for about 10 hours.

b) Placing the raw material (e.g., cellulose) to be chemically treated, preferably degreased and bleached plant fiber in the form of gauze into the mixed solution in the reaction vessel. Maintaining the temperature of the external body above 20° C., preferably near about 30° C.±3° C. Moreover, the method may comprise maintaining the temperature of the internal bladder between about 20° C. and about 30° C., and preferably at about 26° C.±1° C.

c) Decreasing the temperature of the internal bladder to about 20° C.±3° C., and beginning to rotate the reaction vessel for a period of time, preferably about five hours.

d) Allowing cold water to move into the internal bladder so that after a period of time, the temperature may drop to below 20° C., and preferably to about 5° C.±3° C. Allow the solution to react at this decreased temperature for a period of time, preferably about one hour.

e) Adding an appropriate amount of alcohol, preferably 95% ethyl alcohol, and an appropriate amount of chloroacetic acid, into the reaction vessel. After 30 minutes, the temperature in the internal bladder may increase up to a temperature above 20° C., preferably the temperature will move from about 5° C.±3° C. to about 41° C.±3° C. Add an appropriate amount of hydrogen peroxide. Decrease the temperature below 40° C., preferably to about 32° C.±3° C., and allow the reaction to continue for a period of time, preferably about 1.5 hours.

f) Put the material from the reaction vessel into a container, preferably a stainless-steel tub. Add an appropriate amount of alcohol, preferably 70% ethyl alcohol, stir and rise. At that point, dry it up, preferably by centrifugal dewatering.

g) Put the material obtained as above into another container, preferably made of stainless steel, with an appropriate amount of a selected alcohol, preferably 70% ethyl alcohol, then counteract it by adding an acid, preferably Hydrochloric acid, to solution to achieve the desired pH value, preferably a pH value of about 7±0.5.

h) Take out the material and allow it to dry. Preferably one would treat the material one more time or many times as described as above in another container until the solution becomes clear. Allow the material to dry. Optionally one may iron the material to make it flat.

I) Place the dried hemostatic material into a machine to transform the sheets of material into granules or a powder. The machine may pulverize the sheets of material to form a powder, grind the sheets of material into granules, chop the sheets of materials to form flakes or the like.

5. Formation of Hemostatic Gels

In order to form a hemostatic gel from the aforementioned dried (solid) hemostatic material comprising oxidized derivatized esterified cellulose, the solid hemostatic material is combined with a liquid and mixed until a homogenous gel is formed. The liquid may be in the form of sterile water, saline and/or thrombin. The quantity of liquid to the amount of solid hemostatic material is such that the absorption properties are not completely depleted while allowing a flowable gel to be formed that can be dispersed as through a syringe.

By way of example, the ratio of solid hemostatic material to liquid is in a ratio of about 1 gram of solid hemostatic material to about 4 mL of liquid. Thus, to form a hemostatic gel according to the present invention, 1 gram of solid hemostatic material is mixed with 4 mL of saline to produce a saline-based hemostatic hydrocolloid. Similarly, to form a thrombin-based hydrocolloid, 1 gram of solid hemostatic material is mixed with 4 mL of thrombin. In either case, an antibiotic, such as Ancef, can be added to the mixture according to a prescribed dosage. For example, 1 gram of Ancef can be mixed with 1 mL of saline and mixed with 1 gram of solid hemostatic material to form the hemostatic hydrocolloid. Likewise, the aforementioned thrombin-based hydrocolloid can be mixed with 1 mL of saline containing an appropriate dose of an antibiotic, such as 1 gram of Ancef. Such ratios of solid hemostatic material to liquid allows a flowable hydrogel to be formed without causing the hemostatic material to be fully saturated with the liquid. This further allows for the resulting hemostatic gel to be applied to a wound and allows the hemostatic hydrogel to further react with fluids at the wound site to assist blood coagulation.

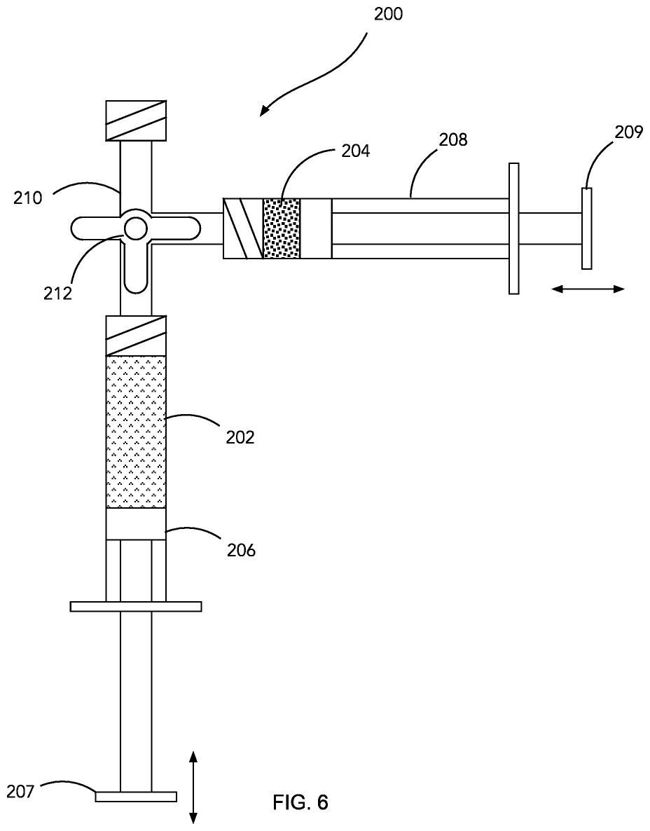

As shown in FIG. 6, a system for forming a hemostatic gel (in the form of a hydrogel), generally indicated at 200 is formed by mixing a liquid 202 with a solid hemostatic material 204 at a time when the hemostatic gel is needed. In FIG. 6, two mixing and dispensing devices in the form of syringes 206 and 208 are interconnected with a three-way stopcock 210. The stopcock 210 includes a valve 212 that is positioned such that flow can pass only between the two syringes 202 and 204. The first syringe 206 contains the liquid 202, such as sterile water or saline. The second syringe 208 contains the hemostatic material 204 in solid form, such as granulated or powdered oxidized derivatized esterified cellulose. By pressing the first syringe plunger, the liquid 202 is forced from the first syringe 206 into the second syringe 208, thereby wetting the hemostatic material 204. This causes the second syringe plunger 209 to extend. Pressing the second syringe plunger 209 then forces the hemostatic material/liquid mixture from the second syringe 208 to the first syringe 206, thus causing the plunger 207 of the first syringe 206 to extend. The plunger 207 of the first syringe 206 is then pressed to force the hemostatic material/liquid mixture through the stopcock 210 back into the second syringe 208. This process is repeated until a visibly homogenous mixture of the hemostatic material/liquid is formed, resulting in the formation of a hemostatic gel. The syringe 206 or 208 containing the resulting hemostatic gel is removed from the stopcock 210 and directly used to inject the hemostatic gel into a wound site to help inhibit bleeding at the wound. Furthermore, by providing the hemostatic gel in a syringe, the hemostatic gel can be easily directed into a wound with the amount of hemostatic gel applied being easily controllable with the syringe.

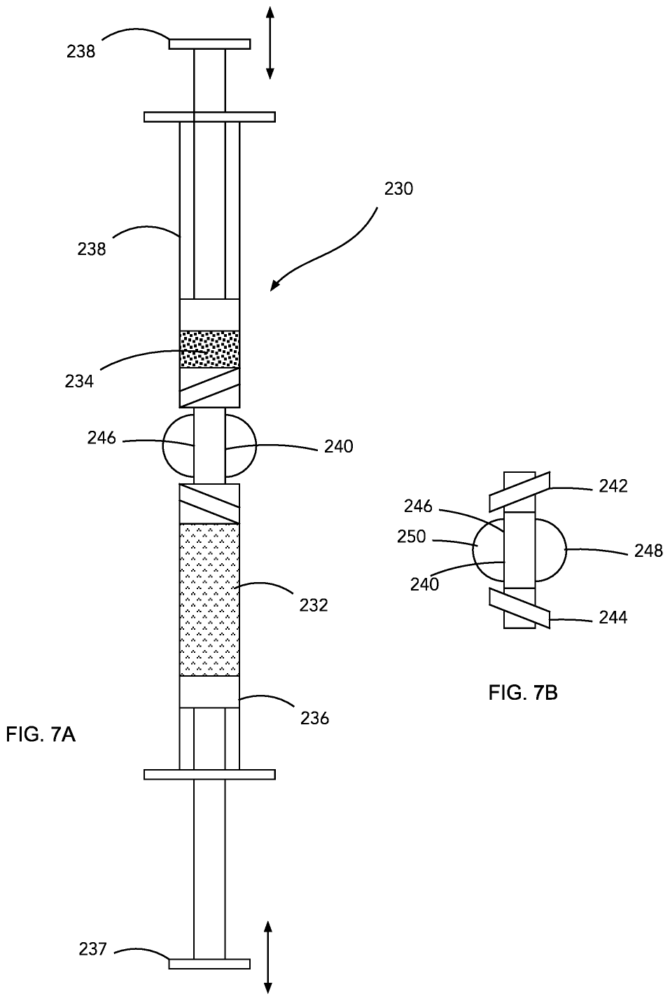

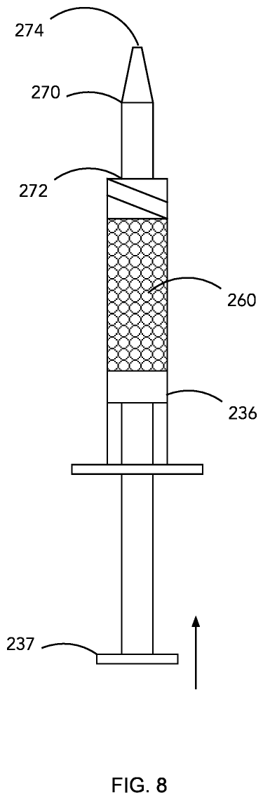

As shown in FIGS. 7A and 7B, a system for forming a hemostatic gel (in the form of a hydrogel), generally indicated at 230 is formed by mixing a liquid 232 with a solid hemostatic material 234 at a time when the hemostatic gel is needed. In FIG. 7A, two syringes 236 and 238 are interconnected with a female-to-female luer lock adapter 240. As shown in FIG. 7B, the luer lock adapter 240 includes a first luer lock end 242 at a first end and a second luer lock end 244 at a second end. A tube portion 246 is in fluid communication with and interconnects the first and second luer lock ends 242 and 244. A pair of oppositely extending finger tabs 248 and 250 are attached to and depend from the tube portion 246 for gripping the luer lock adapter 240 when connecting or removing the syringes 236 and 238. The luer lock adapter 240 is connected to and between the syringes 236 and 238 such that flow can pass only through the luer lock adapter 240 and thus between the two syringes 236 and 238. The first syringe 236 contains the liquid 232, such as sterile water or saline. The second syringe 238 contains the hemostatic material 234 in solid form, such as granulated or powdered oxidized derivatized esterified cellulose. By pressing the first syringe plunger 237, the liquid 232 is forced from the first syringe 236 into the second syringe 238, thereby wetting the hemostatic material 234. This causes the second syringe plunger 239 to extend. Pressing the second syringe plunger 239 then forces the hemostatic material/liquid mixture from the second syringe 238 to the first syringe 236, thus causing the plunger 237 of the first syringe 236 to extend. The plunger 237 of the first syringe 236 is then pressed to force the hemostatic material/liquid mixture through the luer lock adapter 240 back into the second syringe 238. This process is repeated until a visibly homogenous mixture of the hemostatic material/liquid is formed, resulting in the formation of a hemostatic gel 260 as shown in FIG. 8. In order to view the condition of the hemostatic material/liquid until formation of the hemostatic gel 260, the syringes 236 and 238 are formed from a sufficiently transparent plastic material so that their respective contents and the condition thereof can be visibly seen as mixing progresses.

As shown in FIG. 8, the syringe 236 containing the resulting hemostatic gel 260, which has been removed from the adapter 240 (see FIG. 7A), is fitted with a dispensing adapter 270. The dispensing adapter has a luer lock connector at a first end 272 and a dispensing tip 274 at a second opposite end. The first end 272 is coupled to the distal end of the syringe 236 and held together with the luer lock connection. The dispensing adapter 240 comprises a hollow tube that is configured to allow flow of the hemostatic gel therethrough and thus to dispense the hemostatic gel into a wound site as the plunger 237 is depressed relative to the body of the syringe as indicated by the arrow to inhibit bleeding at the wound site. Furthermore, by providing the hemostatic gel in a syringe, the hemostatic gel can be easily directed into a wound with the amount of hemostatic gel applied being easily controllable with the syringe.

The hemostatic gel of the various embodiments exhibits good adherence to wounds such that an adhesive or other material to enhance the adhesive properties of the hemostatic gel is generally not necessary.

6. Formation of Hemostatic Foams

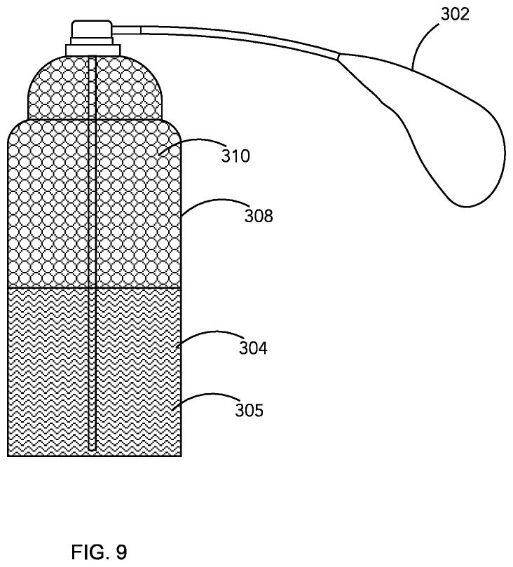

As shown in FIG. 9, in order to form a hemostatic foam 302 from a hemostatic gel 304 comprising oxidized derivatized esterified cellulose, as shown in FIG. 7, the hemostatic gel 304 is combined with a liquid foaming agent 305 and placed in a pressurized container 308 containing a propellant 310. The hemostatic gel 304 and liquid foaming agent 305 are propelled from the container 310 to a desired site on which the hemostatic foam 302 is needed to inhibit bleeding.

7. Use of Additional Hemostatic Agents

Other suitable hemostatic agents that can be employed in various embodiments may include, but are not limited to, clotting factor concentrates, recombinant Factor VIIa, alphanate FVIII concentrate, bioclate FVIII concentrate, monoclate-P FVIII concentrate, haemate P FVIII, von Willebrand factor concentrate, helixate FVIII concentrate, hemophil-M FVIII concentrate, humate-P FVIII concentrate, hyate-C®. Porcine FVIII concentrate, koate HP FVIII concentrate, kogenate FVIII concentrate, recombinate FVIII concentrate, mononine FIX concentrate, and fibrogammin P FXIII concentrate. Such hemostatic agents can be applied to the hemostatic material of this invention in any suitable form (e.g., as a powder, as a liquid, in a pure form, in a suitable excipient, on a suitable support or carrier, or the like).

A single hemostatic agent or combination of hemostatic agents can be employed. Loading levels for the hemostatic agent on the hemostatic material can vary, depending upon, for example, the nature of the selected material and hemostatic agent, the form of the material, and the nature of the wound to be treated. However, in general in the case of hemostatic gauze, a generally preferred weight ratio of hemostatic agent to hemostatic gauze may be from about 0.001:1 or lower to about 2:1 or higher. More preferably, a weight ratio of additional hemostatic agent to hemostatic material may be from about 0.05:1 or lower to about 2:1 or higher. More preferably, a weight ratio from about 0.06:1, 0.07:1, 0.08:1, 0.09:1, 0.10:1, 0.15:1, 0.20:1, 0.25:1, 0.30:1, 0.35, 0.40:1, 0.45:1, 0.50:1, 0.55:1, 0.60:1, 0.65:1, 0.70:1, 0.75:1, 0.80:1, 0.85:1, 0.90:1, or 0.95:1 to about 1:1, 1.1:1, 1.2:1, 1.3:1, 1.4:1, or 1.5:1 may be employed, although higher or lower ratios can be preferred for certain embodiments.

8. Use of Auxiliary Substances in Preparing Hemostatic Materials

In certain embodiments, it can be desirable to utilize the hemostatic gel comprising the described oxidized derivatized esterified cellulose alone as the hemostatic material. However, in other embodiments, other materials such as collagen or other materials may be used in conjunction with the described oxidized derivatized esterified cellulose as a hemostatic material. Other substances that can be utilized in conjunction with the described oxidized derivatized esterified cellulose may include thrombin, fibrinogen, hydrogels, and oxidized cellulose. Other auxiliary substances can also be employed, as will be appreciated by one skilled in the art.

9. Multifunctional Hemostatic Materials

In addition to effectively delivering a hemostatic agent to a wound, in some embodiments, the hemostatic materials comprising oxidized derivatized esterified cellulose can deliver other substances as well. In a particular embodiment, such substances may include medicaments, antibiotics, pharmaceutical compositions, therapeutic agents, and/or other substances producing a physiological effect. The substances can be deposited on the hemostatic material by any suitable method known in the art for depositing a material onto another material or incorporating an agent into a material.

In some embodiments, any suitable medicament, pharmaceutical composition, therapeutic agent, antibiotic or other desirable substance can be incorporated into the hemostatic material comprising the described oxidized derivatized esterified cellulose. The medicaments may include, but are not limited to, anti-inflammatory agents, anti-infective agents, antibiotics, anesthetics, immunosuppressive agents and chemotherapy agents.

Some non-limiting examples of suitable anti-inflammatory agents may include, but are not limited to, nonsteroidal anti-inflammatory drugs (NSAIDs), such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids, such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

Anti-infective agents may include, but are not limited to, anthelmintics (mebendazole), antibiotics including am inoclycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lam ivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim.

Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, rem ifentanil, sufentanil, butorphanol, nalbuphine, tramadol, benzocaine, dibucaine, ethyl chloride, xylocalne, and phenazopyridine.

Chemotherapy agents may include, but are not limited to, adriamycin, alkeran, Ara-C, BiCNU, busulfan, CCNU, carboplatinum, cisplatinum, cisplatinum, daunorubicin, DTIC, 5-FU, fludarabine, hydrea, idarubicin, ifosfamide, methotrexate, mithramycin, mitomycin, mitoxantrone, nitrogen mustard, taxol, velban, vincristine, VP-16, gemcitabine (gemzar), herceptin, irinotecan (camptosar, CPT-11), leustatin, navelbine, rituxan, STI-571, taxotere, topotecan (hycamtin), xeloda (capecitabine), and zevelin.

A variety of other medicaments and pharmaceutical compositions may also be suitable for use in various embodiments of the hemostatic material comprising oxidized derivatized esterified cellulose. These may include cell proliferative agents, such as tretinoin; procoagulants, such as dencichine (2-amino-3-(oxalylamino)-propionic acid); and sunscreens, such as oxybenzone and octocrylene.

Human epidermal growth factor (hEGF) can also be used in certain embodiments. This small molecular weight peptide is a mitogenic protein and may be critical for skin and epidermal regeneration. It may be a small 53 amino acid residue long protein with 3 disulfide bridges. The epidermal growth factor can be used as produced, or can be polymerized prior to use in preferred embodiments. Presence of hEGF can have a positive effect upon skin healing and regeneration.

Other substances which can be used in various embodiments can include, or be derived from, traditional medicaments, agents, and remedies that have known antiseptic, wound healing, and pain relieving properties. These agents may include, but are not limited to, Sanqi (Radix Notoginsent). Another such agent may be Dahuang (Radix Et Rhizoma Rhei). One of its compounds, Emodin, may cause anti-inflammatory effects and can also effectively reduce soft tissue edema. Another agent may include Zihuaddng (Herba Violae), which has been used as an antibiotic agent.

Baiji (Rhizoma Bletillae) has been used as a hemostatic agent and also to promote wound healing for years. It may contain the following substances: (3,3′-di-hydroxy-2′,6′-bis(p-hydroxybenzyl)-5-methoxybibenzy-1); 2,6-bis(p-hydroxybenzyl)-3′,5-dimethoxy-3-hydroxy-bibenzyl); (3,3′-dihydroxy-5-methoxy-2,5′,6-tris(p-hydroxy-benzyl) bibenzyl; 7-dihydroxy-1-p-hydroxybenzyl-2-methoxy-9,10-dihydro-phenanthrene); (4,7-dihydroxy-2-methoxy-9, 10-dihydroxyphenanthrene); Blestriarene A (4,4′-dimethoxy-9,9′,10,10′-tetrahydro[1,1′-biphenanthrene]-2,2′,7,7′-te-trol); Blestriarene B (4,4′-dimethoxy-9,10-dihydro[1,1′-biphenanthrene]-2-,2′,7,7′-tetrol); Batatasin; 3′-O-Methyl Batatasin; Blestrin A(1); Blestrin B(2); Blestrianol A (4,4′-dimethoxy-9,9′,10,10′-tetrahydro]-1′,3-1-biphenanthrene]-2,2′,7,7′-tetraol); Blestranol B (4′,5-dimethoxy-8-(4-hyd-roxybenzyl)-9,9′,10,10′-tetrahydro-[1,3-biphenanthrene]-2,2′,7,7′-tetraol-Blestranol C (4′,5′-dimethoxy-8-(4-hydroxybenzyl)-9,10-dihydro-[′,3-bi-phenanthrene]-2,2′,7,7′-tetraol); (1,8-bi(4-hydroxybenzyl)-4-methoxy-phena-nthrene-2,7-diol); 3-(4-hydroxybenzyl)-4-methoxy-9,10-dihydro-phenanthrene-2,7-diol; (1,6-bi(4-hydroxybenzyl)-4-methoxy-9,10-dihydro-phenanthrene-2,-7-diol; (1-p-hydroxybenzyl-4-methoxyphenanthrene-2,7-diol); 2,4,7-trimethoxy-phenanthrene; 2,4,7-trimethoxy-9,10-dihydrophenanthrene; 2,3,4,7-tetramethoxyphenanthrene; 3,3′,5-trimethoxy-bibenzyl; 3,5-dimethoxybibenzyl; and Physcion.

Rougui (Cortex Cinnamoni) has pain relief effects. It may contain some or all of the following substances: anhydrocinnzeylanine; anhydrocinnzeylanol; cinncassiol A; cinnacassiol A monoacetate; cinncassiol A glucoside; cinnzeylanine; cinnzeylanol; cinncassiol B glucoside; cinncassiol C₁; cinncassiol C₁ glucoside; cinncassiol C₂; cinncassiol C₂; cinncassiol D₁; cinncassiol D₁ glucoside; cinncassiol D₂; cinncassiol D₂glucoside; cinncassiol D₃; cinncassiol D₄; cinncassiol D₄ glucoside; cinncassiol E; lyoniresinol; 3α-O-B-D-glucopyranoside; 3,4,5-trimethoxyphenyl 1-O-β-D-apiofuranosyl-(1→6)-β-D-glucopyranoside; (±)-syringaresinol; cinnamic aldehyde cyclic glycerol 1,3 acetals; epicatechin; 3′-O-methyl-(−)-epicatechin; 5,3′-di-O-methyl-(−)-epicatechin-; 5,7,3′-tri-O-methyl-(−)-epicatechin, 5′-O-methyl-(+)-catechin; 7,4′-di-O-methyl-(+)-catechin; 5,7,4′-tri-O-methyl-(+)-catechin; (−)-epicatechin-3-O-[3-D-glucopyranoside; (−)-epicatechin-8-C-β-D-glucopyranoside; (−)-epicatechin-6-C-β-D-glucopyranoside; procyanidin; cinnamtannin A2, A3, A4; (−)-epicatechin; procyanidins B-1, B-2, B-5, B-7, C-1; proanthocyanidin; proanthocyanidin A-2; 8-C-β-D-glucopyranoside; procyanidin B-2 8-C-β-D-glycopyranoside; cassioside [(4s)-2,4-dimethyl-3-(4-hydroxy-3-hydroxymethyl-1-butenyl)-4-(β-D-glucopyranosyl)methyl-2-cyclohexen-1 -one]; 3,4,5-trimethoxyphenyl-β-D-apiofuranosyl-[(1.f-wdarw.6)-β-D-glucopyranoside; coumarin; cinnamic acid; procyanidin; procyanidin B₂; cinnamoside[(3R)-4-{(2′R,4′S)-2′-hydroxy-4′-(β-1-D-apiofuranoxy-(1→6)-β-D-glucopyranosyl)-2′,6′,6′-trimethyl-cyclohexylidene}-3-buten-2-one]; cinnamaldehyde; 3-2(hydroxyphenyl)-propano-ic acid; O-glucoside; cinnaman A₂; P, S, CI, K, Ca, Ti, Mn, Fe, Cu, Zn, Br, Rb, Sr, and Ba.

Other substances that can be incorporated into the hemostatic material of various embodiments may include various pharmacological agents, excipients, and other substances well known in the art of pharmaceutical formulations . Other pharmacological agents may include, but are not limited to, antiplatelet agents, anticoagulants, ACE inhibitors, and cytotoxic agents. These other substances can include ionic and nonionic surfactants (e.g., Pluronic™, Triton™), detergents (e.g., polyoxyl stearate, sodium lauryl sulfate), emulsifiers, demulsifiers, stabilizers, aqueous and oleaginous carriers (e.g., white petrolatum, isopropyl myristate, lanolin, lanolin alcohols, mineral oil, sorbitan monooleate, propylene glycol, cetylstearyl alcohol), emollients, solvents, preservatives (e.g., methylparaben, propylparaben, benzyl alcohol, ethylene diamine tetraacetate salts), thickeners (e.g., pullulin, xanthan, polyvinylpyrrolidone, carboxymethylcellulose), plasticizers (e.g., glycerol, polyethylene glycol), antioxidants (e.g., vitamin E, vitamin K, vitamin C, calcium), buffering agents, flexible agents (e.g., silicon), antibiotics, low-grade antibiotics (e.g., silver, tetracycline, etc.), and the like.

The following examples may describe this invention in further detail, but these examples shall not be construed as limiting the scope of this invention.

### EXAMPLE 1

As mentioned, the hemostatic material comprising the described oxidized derivatized esterified cellulose may be made in a variety of methods. Nevertheless, one non-limiting example of a method for making the hemostatic material may comprise:

1) Activating Treatment:

a) Placing two liters of sodium hydroxide, two liters of sodium carbonate, and one liter of sodium hypochlorite in to the internal bladder of a reaction vessel, then adding in an appropriate amount of pure water and stirring until the ingredients are totally dissolved and a pH value of about 8 to 9.5 is achieved. Then, pouring 60 liters of 95% ethyl alcohol in to the internal bladder and mix. Then turn on the stainless steel heater and keep the temperature of the internal bladder between about 25° C. and about 28° C. and hold for 10 hours.

b) Put about 80 meters of clinical use gauze made from cellulose into the mixed solution in the reaction vessel. At this point, the temperature of the external body should be 30° C.±3° C. Additionally, the temperature of the internal bladder should be 26° C.±1° C.

c) Decrease the temperature of the internal bladder to 20° C.±3° C., and begin to rotate the reaction vessel for about three to about five hours.

d) Allow cold water from a refrigerator to move into the internal bladder with a temperature of 20° C.±3° C., after 30 minutes the temperature will drop to 5° C.±3° C. Allow this reaction to occur for one hour.

2) Oxidizing Treatment

a) Add about 60 liters of 95% ethyl alcohol and 12 bottles of chloroacetic acid into the reaction vessel. Then let in water with the temperature at about 45° C. After 30 minutes the temperature in the internal bladder may go up from 5° C.±3° C. to 41° C.±3° C. Add one bottle of hydrogen peroxide, decrease the temperature to 32° C.±3° C., and allow the reaction to continue for about 1.5 hours.

3) Rinsing and Drying Up

a) Put the gauze form the reaction vessel into a stainless-steel tub, add in 60 kg 70% ethyl alcohol, stir and rise. Then dry the gauze up by centrifugal dewatering.

b) Put the gauze obtained as above into another stainless-steel tub with 60 kg 70% ethyl alcohol; counteract it by adding into Hydrochloric acid to achieve the pH value of 7±0.5.

c) Take out the gauze, dry it up, and treat the gauze one more time or many times as described above in another stainless-steel tub until the solution becomes clear. Then take out the gauze, dry it up, and make it flat by ironing.

d) Dry the rinsed gauze up in a dryer. Turn on the power switch, press on the drying button, the dryer begins to run and removes the unwanted ethyl alcohol form the gauze.

4) Sterilizing and Ironing Out

for drying and ironing. The rolling of the rollers may make the gauze go through and as well as continue to dry up the gauze and iron out the gauze and so the gauze may come out flat before being scrolled up.

5) Pulverizing

a) insert a sheet of the gauze into a pulverizing machine and grind the gauze into a powder.

6) Kit

a) placing 1 gram of the hemostatic powder into a first syringe.

b) drawing 4 mL of saline into a second syringe.

c) providing a mixing stopcock with the first and second syringes that can be used to combine the two syringes for mixing the saline with the hemostatic powder when needed.

Having described these aspects of the invention, it is understood that the invention provides a new kind of soluble hemostatic materials and it can be made in the industry simply and economically. It is also understood that the invention defined by the appended claims is not to be limited by particular details set forth in the above description, as many apparent variations thereof are possible without departing from the spirit or scope thereof.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A method of forming a hemostatic hydrocolloid for dispensing into a wound site, comprising:
providing a polymer of oxidized derivatized esterified cellulose in solid form in a first mixing and dispensing device, the polymer of oxidized derivatized esterified cellulose comprising a chain of monomers, wherein, for a first plurality of the monomers in the chain: R is —OCH₂(COO)CH₂CH₃, R1 is —OCH₂(COO)CH₂CH₃, and R2 is —CH₂OCH₂(COO)CH₂CH₃; and wherein, for a second plurality of monomers in the chain: R is —OCH₂(COO)CH₂CH₃, R1 is —OCH₂(COO)CH₂CH₃, and R2 is —(COO)CH₂CH₃;
providing a liquid in a second mixing and dispending device;
connecting the first mixing and dispending device to the second mixing and dispensing device to allow flow of the liquid between the first and second dispensing and mixing devices; and
dispensing the liquid into the polymer of oxidized derivatized esterified cellulose in solid form;
repeatedly mixing the liquid and polymer of oxidized derivatized esterified cellulose between the first and second mixing and dispensing devices until a hemostatic gel is formed.

2. The method of claim 1, further comprising connecting an adapter between the first and second mixing and dispensing devices, the adapter configure to allow flow of the liquid between the first and second mixing and dispensing devices.

3. The method of claim 2, wherein the adapter comprises a three-way stopcock.

4. The method of claim 3, wherein the stopcock includes a valve that is positioned so that flow can pass only between the first and second mixing and dispensing devices.

5. The method of claim 1, wherein the liquid comprises sterile water or saline.

6. The method of claim 1, wherein the polymer of oxidized derivatized esterified cellulose is in granules, particles, pieces or in granulated or powdered form.

7. The method of claim 1, wherein the first dispensing and mixing device comprises a first syringe and the second dispensing and mixing device comprises a second syringe.

8. The method of claim 7, further comprising pressing a second plunger of the second syringe to force the liquid from the second syringe into the first syringe, thereby causing a first syringe plunger of the first syringe to extend and wetting the polymer of oxidized derivatized esterified cellulose to form a hemostatic material/liquid mixture.

9. The method of claim 8, further comprising pressing the second syringe plunger to force the hemostatic material/liquid mixture from the second syringe into the first syringe and causing the first syringe plunger of the first syringe to extend.

10. The method of claim 9, further comprising pressing the first syringe plunger to force the hemostatic material/liquid mixture from the first syringe into the second syringe and causing the second syringe plunger of the second syringe to extend and repeatedly alternating pressing the second syringe plunger and first syringe plunger until a visibly homogenous mixture of the hemostatic material/liquid is formed, resulting in the formation of a hemostatic gel.

11. The method of claim 10, further comprising removing the first or second syringe that contains the hemostatic gel from the adapter and using the selected syringe to inject the hemostatic gel into a wound site to inhibit bleeding.

12. The method of claim 2, wherein the adapter comprises a female-to-female luer lock adapter.

13. A system for forming a hemostatic hydrocolloid, comprising:
a polymer of oxidized derivatized esterified cellulose comprising a chain of monomers, wherein, for a first plurality of the monomers in the chain: R is —OCH₂(COO)CH₂CH₃, R1 is —OCH₂(COO)CH₂CH₃, and R2 is —CH₂OCH₂(COO)CH₂CH₃; and wherein, for a second plurality of monomers in the chain: R is —OCH₂(COO)CH₂CH₃, R1 is —OCH₂(COO)CH₂CH₃, and R2 is —(COO)CH₂CH₃; and
a water-based liquid mixed with the polymer of oxidized derivatized esterified cellulose forming a hemostatic gel.

14. The system of claim 13, further comprising a first mixing and dispensing device containing the polymer of oxidized derivatized esterified cellulose and a second mixing and dispensing device containing the water-based liquid.

15. The system of claim 12, further comprising an adapter coupled between the first and second mixing and dispensing devices, the adapter configure to allow flow of the liquid between the first and second mixing and dispensing devices.

16. The system of claim 14, wherein the adapter comprises a three-way stopcock.

17. The system of claim 15, wherein the stopcock includes a valve that is positioned so that flow can pass only between the first and second mixing and dispensing devices until the hemostatic gel is formed and then is positioned to allow flow of the hemostatic gel out of the three-way stop cock.

18. The system of claim 13, wherein the polymer of oxidized derivatized esterified cellulose is in the form of granules, particles, pieces or in granulated or powdered form.

19. The system of claim 14, wherein the first dispensing and mixing device comprises a first syringe and the second dispensing and mixing device comprises a second syringe, whereby pressing a second plunger of the second syringe forces the liquid from the second syringe into the first syringe, thereby causing a first syringe plunger of the first syringe to extend and wetting the polymer of oxidized derivatized esterified cellulose to form a hemostatic gel and whereby pressing the second syringe plunger forces the hemostatic gel from the second syringe into the first syringe and causing the first syringe plunger of the first syringe to extend.

20. The method of claim 14, wherein the adapter comprises a female-to-female luer lock adapter.
